(19) United States
(12) Patent Application Publication
(10) Pub. No.: US 2025/0185700 A1
(43) Pub. Date: Jun. 12, 2025

(54) A STABILIZER COMPOSITION COMPRISING MICROCRYSTALLINE CELLULOSE

(71) Applicants: DUPONT NUTRITION BIOSCIENCES APS, Kongens Lyngby (DK); NUTRITION & BIOSCIENCES USA 1, LLC, Rochester, NY (US); DUPONT NUTRITION USA, INC., Wilmington, DE (US)

(72) Inventors: Sampson ANANKANBIL, Aarhus N (DK); Britta HUEBNER-KEESE, Bomlitz (DE); Simon EUSTACE, Lincoln University, PA (US)

(21) Appl. No.: 18/273,221

(22) PCT Filed: Jan. 24, 2022
(86) PCT No.: PCT/US2022/013503

§ 371 Date: Jul. 19, 2023

Related U.S. Application Data
(60) Provisional application No. 63/141,014, filed on Jan. 25, 2021.

(51) Int. Cl. A23L 29/256; A23C 9/154; A23C 11/04; A23L 2/62; A23L 17/60; A23L 29/262; A23P 10/25; A23P 30/25

(52) U.S. Cl. CPC A23L 29/256; A23C 9/154; A23C 11/04; A23L 2/62; A23L 17/60; A23L 29/262; A23P 10/25; A23P 30/25

(57) ABSTRACT

The present invention relates to a stabilizer composition comprising colloidal microcrystalline cellulose coprocessed with unrefined and unmodified red seaweed flour derived from a red seaweed of the class Rhodophyta.

### FIELD OF INVENTION

The present invention is directed to colloidal microcrystalline compositions produced by combining a coprocessed mixture of microcrystalline cellulose and a flour derived from a seaweed of the class Rhodophyta, and their use as stabilizers for edible products.

### BACKGROUND OF THE INVENTION

Microcrystalline cellulose, also known and referred to herein as “MCC”, is hydrolyzed cellulose. MCC powders and gels are commonly used in the food industry to enhance the properties or attributes of a final food product. For example, MCC has been used as a binder and stabilizer in a wide variety of consumable products such as food applications, including in beverages, as a gelling agent, a thickener, a fat substitute, and/or non-caloric filler, and as a suspension stabilizer and/or texturizer. MCC has also been used as a binder and disintegrant in pharmaceutical tablets, as a suspending agent in liquid pharmaceutical formulations, and as a binder, disintegrant, and processing aid, in industrial applications, in household products such as detergents and/or bleach tablets, in agricultural formulations, and in personal care products such as dentifrices and cosmetics. An important application for colloidal MCC is stabilization of suspensions, e.g., suspensions of solid particles in low viscosity liquids; and, more specifically, suspension of solids in milk, e.g., cocoa particles in chocolate milk.

MCC may be modified for the above-mentioned uses by subjecting hydrolyzed MCC aggregated crystallites, in the form of a high solids aqueous mixture, commonly known as “wetcake”, to an attrition process, e.g., extrusion, that substantially subdivides the aggregated cellulose crystallites into more finely divided crystallite particles. To prevent hornification, a protective hydrocolloid may be added before, during, or following attrition, but before drying. The protective hydrocolloid, wholly or partially, screens out the hydrogen bonds or other attractive forces between the smaller sized particles to provide a readily dispersible powder. Colloidal MCC will typically form stable suspensions with little to no settling of the dispersed solids. Carboxymethyl cellulose is a common hydrocolloid used for these purposes (see for example U.S. Pat. No. 3,539,365 (Durand et al.) and the colloidal MCC products sold under the brand names AVICEL® and GELSTAR® by FMC Corporation. Many other hydrocolloids have been tried to co-process with MCC, such as starch, in U.S. Pat. App. 2011/0151097 (Tuason et al.)

There is a growing consumer interest for foods and beverages that are produced using methods, and ingredients retaining naturalness of their raw material, and absent of chemically modified components. This consumer approach has been partly responded to by the industry offering reformulated or new products, in which some food additives, particularly preservatives, colors, and flavors have been successfully eliminated, or replaced with more positively recognised alternative ingredients and food additives of natural origin.

Because of the nature of its processing, CMC has recently come under attack for not being a “clean label” component, although still considered safe by regulatory authorities. As such, attempts have been made to replace CMC with polysaccharides from various plant sources. This has proven challenging, however since each polysaccharide has its own unique structure and it has been difficult to predict their functionality. Many polysaccharides have not been found effective for making dispersion stable MCCs at least partially due to a lack of transfer of sufficient mechanical force to the MCC aggregates and polysaccharides during attrition. One attempt to mitigate the problem has been to use multivalent salts such as calcium chloride (see for example U.S. Pat. No. 7,462,232 B2, to Tuason et al).

There is a need therefore to develop a colloidal MCC composition useful for the stabilization of liquids that contains ingredients recognized by consumers and regulators to be natural products, and that may be effectively attrited without the addition of attrition acids such as salts or acids and avoiding the presence of CMC.

### SUMMARY OF THE INVENTION

The present inventors have met the stated need, by providing a coprocessed colloidal composition that can be effectively attrited without carboxymethyl cellulose and/or attrition aids such as salts or acids, and that can be dispersed easily in consumable products such as food, beverage, pharmaceutical, industrial, and many other products; including, cool/ambient milk products, e.g., chocolate milk or creamers, without the use of sequestrants.

Accordingly, the present invention relates to a stabilizer composition comprising colloidal microcrystalline cellulose coprocessed with unrefined and unmodified red seaweed flour derived from a red seaweed of the class Rhodophyta.

It was surprisingly found possible to provide a stabilizer composition made with unrefined and unmodified red seaweed flour without significant loss of functionality, e.g. reduction of sedimentation in a beverage, compared to a stabilizer comprising colloidal MCC co-attrited with CMC or with refined carrageenan cf. the Example below).

In another aspect, the invention relates to a process for preparing the stabilizer composition of any one of claims 1-10, comprising the steps of
(a) blending a microcrystalline cellulose wetcake with red seaweed flour derived from a red seaweed of the class Rhodophyta,
(b) coprocessing the blend of step (a) in the absence of an attrition aid to form an extrudate, and
(c) drying and optionally milling the extrudate to a powder.

### DESCRIPTION OF EMBODIMENTS

### Definitions

As used herein, “aggregated MCC” means MCC prior to attrition; “attrited MCC” means MCC after attrition; and, “colloidal MCC” means MCC, after attrition in which the D₅₀ of at least 19% by volume of the MCC particles is about 0.1 microns as measured by dynamic light scattering.

The term “attrition aid” means a reagent added to an aggregated MCC composition that facilitates attrition, particularly extrusion. The attrition aid may typically be a salt or an acid.

The term “dispersion stability” or “dispersion stable”, as used herein, means that the colloidal MCC particles themselves disperse uniformly in liquids, e.g., an aqueous medium, without vigorous agitation forming a suspension having a homogenous appearance without significant separating, aggregating or settling of the particles.

The term “suspension stability”, as used herein, means that when the colloidal MCC particles are dispersed in a liquid, e.g., aqueous medium, milk, etc., containing insoluble components other than the MCC particles, e.g., cocoa, calcium, etc., those particles are effectively suspended forming a stabilized suspension having a homogenous appearance without significant separating, aggregating, or settling of the insoluble particles.

The terms “coprocessing” and “coprocessed” are used interchangeably to mean a process that effectively reduces the size of at least some if not all of the particles to a colloidal size. The term “coprocessing” refers to application of high shear forces to an admixture of the MCC and at least one polysaccharide. Suitable processing conditions may be obtained, for example, by co-extruding, milling, or kneading. Coprocessing is also referred to in the literature as “co-attrition”.

### Microcrystalline Cellulose

The present invention makes use of hydrolyzed microcrystalline cellulose. Microcrystalline cellulose (MCC) is a white, odorless, tasteless, relatively free flowing, crystalline powder that is virtually free from organic and inorganic contaminants. It is a purified, partially depolymerized cellulose obtained by subjecting alpha cellulose obtained as a pulp from fibrous plant material to hydrolytic degradation typically with mineral acids. It is a highly crystalline particulate cellulose consisting primarily of crystalline aggregates which are obtained by removing amorphous regions (or paracrystalline regions) of a cellulosic fibril. MCC is used in a variety of applications including foods, nutraceuticals, pharmaceuticals and cosmetics.

Any microcrystalline cellulose may be employed in the compositions of the present invention. Suitable feedstocks include, for example, wood pulp such as bleached sulfite and sulfate pulps, corn husks, bagasse, straw, cotton, cotton linters, flax, hemp, ramie, fermented cellulose, etc. Microcrystalline cellulose may be produced by treating a source of cellulose, preferably alpha cellulose in the form of pulp from fibrous plant materials, with a mineral acid, preferably hydrochloric acid. The acid selectively attacks the less ordered regions of the cellulose polymer chain thereby exposing and freeing the crystalline sites which form crystallite aggregates which constitute the microcrystalline cellulose. These are then separated from the reaction mixture and washed to remove degraded by-products. The resulting wet mass, generally containing 40 to 75 percent moisture, is referred to in the art by several names, including hydrolyzed cellulose, hydrolyzed cellulose wetcake, level-off DP cellulose, microcrystalline cellulose wetcake or simply wetcake. Preferably, the aggregated MCC is acid hydrolyzed and 25-60% wt. in water.

When the wetcake is dried and freed of water the resulting product, microcrystalline cellulose, is a white, odorless, tasteless, relatively free-flowing powder, insoluble in water, organic solvents, dilute alkalis and acids. For a description of microcrystalline cellulose and its manufacture see U.S. Pat. No. 2,978,446. The patent describes its use as a pharmaceutical excipient, particularly as a binder, disintegrant, flow aid, and/or filler for preparation of compressed pharmaceutical tablets.

### Seaweed Flour

As discussed herein, the seaweed flour is derived from seaweeds that taxonomically belong to the class of Rhodophyta. Such seaweeds may be referred to as ‘red seaweed’. Examples of suitable seaweeds belong to the genera consisting of Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus.

The seaweed flour is derived from seaweed of the class of Rhodophyta. By the term “derived from” it is meant that the ingredient is obtained from seaweed of the class of Rhodophyta. The seaweed flour co-attrited with MCC is “unrefined” and “unmodified” which is intended to mean without isolating, purifying or chemically modifying individual components in the seaweed such as carrageenans. The seaweed flour derived from seaweed of the class of Rhodophyta is however minimally treated to obtain it from the seaweed, for example the treatment may typically comprise washing, drying and grinding.

In one aspect, the seaweed is at least of the genus Eucheuma or Kappaphycus. As will be understood by one skilled in the art, the genus Eucheuma has recently been renamed as Kappaphycus. Therefore, references to the genus Eucheuma may be equivalent to the genus Kappaphycus. In one aspect, the seaweed is at least of the genus Eucheuma. In one aspect, the seaweed is at least of the genus Kappaphycus.

In one aspect, the seaweed is at least of the species Eucheuma striatum, Kappaphycus striatus (also known as Kappaphycus striatum), Eucheuma alvarezii, Kappaphycus alvarezii, or a combination thereof. As discussed above, the genus Eucheuma may be equivalent to the genus Kappaphycus. Therefore, references to Euchema striatum may be equivalent to Kappaphycus striatus, and references to Euchema alvarezii may be equivalent to Kappaphycus alvarezii. In one aspect, the seaweed is at least of the species Euchema striatum/Kappaphycus striatus, Euchema alvarezii/Kappaphycus alvarezii, or a combination thereof. In one aspect, the seaweed is a combination of the species Eucheuma striatum/Kappaphycus striatus and Eucheuma/Kappaphycus alvarezii, this combination may be known commercially as Eucheuma/Kappaphycus cottonii.

The seaweed flour is obtained from red seaweeds of the class of Rhodophyta and therefore the flour may be referred to as “red seaweed flour”. The term “red seaweed flour” is to be understood as a description of a flour-like product derived from red seaweed of the Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia and Chondracanthus genera.

The seaweed flour included in the present stabilizer composition may be prepared by any suitable process. Thus, the seaweed flour may be obtained by a process comprising the steps of:
(a) providing seaweed of the class of Rhodophyta,
(b) drying the seaweed of step (a),
(c) rehydrating the dried seaweed at a temperature of from 20° C. to 85° C., optionally in the presence of a salt solution (such as NaCl) at a pH that is lower than 9.5,
(d) separating the rehydrated seaweed of step (c) from the solution;
(e) drying the product of step (d), and
(f) optionally milling the dried product of step (e) to form the food ingredient.

For example, the seaweed flour may be prepared by a process as described in International Patent Publication No. WO 2020/242859.

The seaweed flour present in the stabilizer composition of the present invention may be treated to reduce the number of micro-organisms. Thus, the seaweed flour may be heat treated or pasteurized to reduce the number of micro-organisms.

### Carrageenan

As discussed above, the seaweed flour derived from seaweed of the class of Rhodophyta will contain carrageenan. Carrageenan refers to a family of linear sulfated polysaccharides that are extracted from red edible seaweeds. Carrageenan is a high-molecular-weight polysaccharide made up of repeating galactose units and 3,6 anhydrogalactose (3,6-AG), both sulfated and non-sulfated. The units are joined by alternating α-1,3 and ß-1,4 glycosidic linkages. Carrageenan is widely used in the food and other industries as thickening or stabilizing agents. There are three main commercial classes of carrageenan: kappa, iota and lambda carrageenan.

These three varieties differ in their degree of sulfation. Kappa carrageenan has one sulfate group per disaccharide, iota carrageenan has two, and lambda carrageenan has three. When used in food products, carrageenan has the EU additive E numbers E407 or E407a when present as “processed eucheuma seaweed”.

To obtain a stabilizer composition with advantageous properties, it is generally preferred that the red seaweed flour is derived from a seaweed species of the class Rhodophyta that is rich in kappa-carrageenan and low in iota-carrageenan. Kappa carrageenan forms strong, rigid gels in the presence of potassium ions, and reacts with dairy proteins. It is sourced mainly from Kappaphycus alvarezii/cottonii. In the red seaweed flour, the kappa-carrageenan may be present together with one or more other carrageenans, notably with minor amounts of iota-carrageenan, or if the red seaweed flour is sourced from a seaweed of the species Gigartina atropurpurea or another species of Gigartina, it may be a copolymer of kappa-and iota-carrageenan, e.g. kappa-2-carrageenan as described in EP 1628643 B1.

In one aspect, the red seaweed flour contains kappa-carrageenan in an amount of from 25-75 wt. % based on the dry weight of the red seaweed flour. In another aspect, the red seaweed flour contains kappa-carrageenan in an amount of from 40 to 70 wt. % based on the total weight of the food ingredient. The content of iota-carrageenan in the red seaweed flour is, on the other hand, preferably less than 10 wt. % based on the dry weight of the red seaweed flour.

In one aspect the weight average molecular weight of the kappa-carrageenan present in the red seaweed flour is from 900 to 2000 kDa before co-attrition of the red seaweed flour with MCC. Preferably, the weight average molecular weight of the kappa-carrageenan present in the red seaweed flour before co-attrition with MCC is from 1000 to 1500 kDa.

### Processing Methods

The hydrolyzed MCC and red seaweed flour seaweed flour are typically coprocessed in the absence of an attrition aid to form the coprocessed composition wherein the MCC particles are at least partially coated by one or more components of the red seaweed flour, notably the kappa-carrageenan. Processing methods are common and well known in the art (see for example US Patent Application 2013/0090391 and U.S. Pat. No. 9,828,493 which are hereby incorporated by reference. The methods include preparing an aggregate microcrystalline cellulose of between about 25% and 60% wt. solids. The composition typically comprises MCC and red seaweed flour in a weight ratio between 70:30 and 90:10, preferably between 80:20 and 85:15.

In one embodiment, the coprocessed stabilizer composition has an initial viscosity of 50-1000 mPa·s when measured as a 2.6% by weight dispersion in deionized water on a Brookfield RV viscometer, spindle #3, at 20 rpm and 20° C. In one embodiment, the coprocessed composition has a 24-hour viscosity of 150-5000 mPa·s when measured as a 2.6% by weight dispersion in deionized water on a Brookfield RV viscometer, spindle #3, at 20 rpm and 20° C.

Attrition may be accomplished by extrusion, for example or with other mechanical devices including, e.g., refiners, planetary mixers, colloidal mills, beat mills, kneaders, and grinders that can provide effective shearing force. However, as particle size is reduced, the individual particles tend to agglomerate or hornify upon drying, a result that is undesirable because it impedes dispersion of the individual particles.

The extrudate can be dried or be dispersed in water to form a slurry. The slurry can be homogenized and dried, preferably spray dried. Before drying, an additional amount of red seaweed flour may be wet blended with the extrudate. The amount of additional red seaweed flour may be in the range of 2-20% by total weight of the extrudate and additional seaweed flour. Alternatively, the additional amount of red seaweed flour may be dry blended with the extrudate after drying and milling. Drying processes other than spray drying include, for example, fluidized bed drying, drum drying, bulk drying, and flash drying. Dry particles formed from the spray drying can be reconstituted in a desired aqueous medium or solution to form the compositions, edible food products, pharmaceutical applications, and industrial applications described herein.

Effectiveness of the attrition can be assessed through measuring the viscosity of the mixture of MCC and seaweed flour through the attrition as compared to the viscosity of the mixture of MCC and seaweed flour without the attrition. During an attrition, strong mechanical shear forces not only break down aggregated MCC particles but also introduce a mixing action to spread seaweed flour components such as kappa-carrageenan molecules around the reduced MCC particles. Furthermore, water molecules in between the MCC particles and seaweed flour are squeezed out to bring MCC particles and seaweed flour components into a close contact. Eventually, certain portion on the surface of MCC particles is forced to bond certain segment of kappa-carrageenan chains through molecular interaction force, for instance, the hydrogen bond. In such a manner, the MCC particles act as the node points of kappa-carrageenan network, like crosslinking of kappa-carrageenan, leading to the increase in the viscosity of the mixture of MCC particles and seaweed flour.

### Applications

The stabilizer compositions of the invention may be used in a variety of suitable food, pharmaceutical, nutraceutical and industrial applications including in cosmetic products, personal care products, consumer products, agricultural products, or in chemical formulations and in paint, polymer formulations.

Some examples in pharmaceutical applications include liquid suspending agents and/or emulsions for drugs; nasal sprays for drug delivery where the colloidal MCC gives increased residence and bioavailability; controlled release agents in pharmaceutical applications; and re-constitutable powders which are dry powders mixtures containing drugs which can be made into a suspension by adding water and shaking by-hand; topical drug applications, and various foams, creams, lotions for medical uses, including compositions for oral care such as toothpaste, mouthwash and the like.

Some examples in nutraceutical applications include delivery systems for various nutraceutical ingredients and dietary supplements. Examples in industrial applications include various suspensions, thickeners, which can be used in foams, creams, lotions and sun-screens for personal care applications; suspending agents, which can be used with pigments and fillers in ceramics, or used in colorants, optical brighteners, cosmetics, and oral care in products such as toothpaste, mouthwash and the like; materials such as ceramics; delivery systems for pesticides including insecticides; delivery of herbicides, fungicides, and other agricultural products, and paints, and various chemical or polymer suspensions. One particular example is an industrial wash fluid, containing oxidizing or bleach agents, which demand strong and stable suspension systems

In particular, the stabilizer composition of the present invention may be used in a variety of food products including emulsions, beverages, sauces, soups, syrups, dressings, films, dairy and non-dairy milks and creamers, frozen desserts, cultured foods, bakery fillings, and bakery cream, where it meets the consumer preference for natural food ingredients. It may also be used for the delivery of flavoring agents and coloring agents. The edible food products can additionally comprise diverse edible material and additives, including proteins, fruit or vegetable juices, fruit or vegetable pulps, fruit-flavored substances, or any combination thereof.

These food products can also include other edible ingredients such as, for example, mineral salts, protein sources, acidulants, sweeteners, buffering agents, pH modifiers, stabilizing salts, or a combination thereof. Those skilled in the art will recognize that any number of other edible components may also be added, for example, additional flavorings, colorings, preservatives, pH buffers, nutritional supplements, process aids, and the like. The additional edible ingredients can be soluble or insoluble, and, if insoluble, can be suspended in the food product. Routine adjustment of the composition is fully within the capabilities of one having skill in the art and is within the scope and intent of the present invention. These edible food products can be dry mix products (instant sauces, gravies, soups, instant cocoa drinks, etc.), low pH dairy systems (sour cream/yogurt, yogurt drinks, stabilized frozen yogurt, etc.), baked goods, and as a bulking agent in non-aqueous food systems and in low moisture food systems.

Proteins suitable for the edible food products incorporating the stabilizer compositions include food proteins and amino acids, which can be beneficial to mammals, birds, reptiles, and fish. Food proteins include animal or plant proteins and fractions or derivatives thereof. Animal derived proteins include milk and milk derived products, such as heavy cream, light cream, whole milk, low fat milk, skim milk, fortified milk including protein fortified milk, processed milk and milk products including superheated and/or condensed, sweetened or unsweetened skin milk or whole milk, dried milk powders including whole milk powder and Nonfat Dry Milk (NFDM), casein and caseinates, whey and whey derived products such as whey concentrate, delactosed whey, demineralized whey, whey protein isolate. Egg and egg-derived proteins may also be used. Plant derived proteins include nut and nut derived proteins, sorghum, legume and legume derived proteins such as soy and soy derived products such as untreated fresh soy, fluid soy, soy concentrate, soy isolate, soy flour, and rice proteins, and all forms and fractions thereof. Food proteins may be used in any available form, including liquid, condensed, or powdered. When using a powdered protein source, however, it may be desirable to pre-hydrate the protein source prior to blending with stabilizer compositions and juice for added stability of the resulting beverage. When protein is added in conjunction with a fruit or vegetable juice, the amount used will depend upon the desired end result.

It should also be noted that the food/beverage compositions may be processed by heat treatment in any number of ways. These methods may include, but are not limited to, Low Temperature Long Time (LTLT), High Temperature Short Time (HTST), Ultra-High Temperature (UHT) and Extended Shelf Life (ESL) processes. These beverage compositions may also be retort processed, either by rotary retort or static retort processing. Some compositions, such as juice-added or natural or artificially flavored soft drinks may also be cold processed. Many of these processes may also incorporate homogenization or other high shear/high compression methods. There may also be co-dried compositions, which can be prepared in dry-mix form, and then conveniently reconstituted for consumption as needed. The resulting beverage compositions may be refrigerated and stored for a commercially acceptable period of time. In the alternative, the resulting beverages may be stored at room temperature, provided they are filled under aseptic conditions.

The described compositions can act as stabilizers suitable for use in the beverage industry. The compositions, after drying to powder form, can be mixed with an aqueous solution to form a colloidal mixture that, in some embodiments, can maintain its colloidal properties for a long period of time. Some of the edible food products are beverages, protein and nutritional beverages, mineral fortified beverages, dairy-based beverages, and non-dairy based beverages including, but not limited to, those that are heat treated, for example, by pasteurization, ultra-pasteurization, or retort processes.

The typical concentrations of the stabilizer of the present invention used in the above beverage products can range from 0.05% to about 3.5% by wt. of total products, and in some instances 0.2 to 2.0% by wt. of total products.

In particular the compositions of the invention are well suited for stabilization of beverages, particularly dairy milk beverages or plant protein beverages, or as stabilizers in diary or non-dairy creamers. For these applications, the present stabilizer composition may be present in an amount of 0.1-0.5% by weight of the beverage or creamer, preferably in an amount of 0.20-0.35% by weight of the beverage or creamer.

### EXAMPLES

### Preparation of Colloidal MCC Co-Attrited with Red Seaweed Flour

The MCC wetcakes used in following examples were obtained via acid hydrolysis of a prehydrolyzed hardwood pulp (Sulfatate™, available from Rayonier Inc.). The wetcake was prepared for attrition by mixing aggregated MCC at 43.05% wt. total solids, with refined Na-iota carrageenan, red seaweed flour derived from E. spinosum, red seaweed flour derived from E. cottonii or carboxymethyl cellulose as follows:

All ingredients were mixed in a 12-quart bowl on a Hobart A120 mixer (Model No. ML 38904). The wetcake was first loaded in the Hobart mixer bowl. The beater/paddle was then assembled to rotate at lowest setting. Other ingredients such as sodium carbonate were also added to the mixer. The beater/paddle rotation speed was progressively increased to the highest setting until a visually uniform admixture was achieved. This typically took 3-5 minutes. Then, the respective hydrocolloids were mixed in for 3-5 minutes in the Hobart mixer bowl. Afterwards, the admixture was fed into an extruderand processed for a number of times. The extrusion performance was monitored by reading the torque on an attached amperage meter; measuring the temperature of extrudate; and, observing the texture of extrudates. Higher amperage meter readings, hotter extrudate, and firmer extrudate, indicated more effective co-attrition. A simple examination of the extrudates may be performed by measuring the viscosities of the wetcake mixtures slurried in deionized water and by studying dispersion of MCC crystals in the slurries microscopically. Ultimately, the exemplary extrudates were dried into a powder form by slurrying in deionized water before being dried.

### Sample Dispersion Preparation and Viscosity Measurements

Sample dispersions for initial and 24-hour viscosity measurements were prepared in a 700G Waring blender (Model WF2212112) by Waring Commercial with a glass 4 cup bowl size. The speed of rotation blade was adjusted by an autotransformer. Each dispersion sample size was set at 600 g. Samples were introduced to the center of a deionized water vortex at approximately 30 volts. After loading samples, the lid was placed on the bowl. Pre-mixing took about 15 seconds. Voltage of the autotransformer was then increased to 115 volts for 2 minutes. The viscosity of the prepared dispersion was measured promptly using a Brookfield RV viscometer, spindle #3, at 20 rpm and 20° C. This viscosity measurement is termed as initial viscosity. After the initial viscosity measurements, the dispersions were allowed to sit on a bench without disturbance for 24 hours in a closed jar and thereafter the viscosities were measured again.

### Dynamic Rheological Measurements of Sample Dispersions

For dynamic rheological measurements a sample dispersion was prepared that had a solids content of 2.6 wt.-%, based on the total weight of the dispersion. The dynamic moduli (elastic/storage modulus, G′ and loss/viscous modulus, G″) were determined on a HAAKE MARS II rheometer equipped with a UTC temperature controller and a MARS II control unit. The measurement was made on two parallel plates consisting of a 60-mm stainless steel stationary plate, and a 35 mm stainless steel plate (PP35Ti) as rotor. The temperature for the measurement was 20° C. and the gap size between plates set at 1.0 mm. Tan & was determined as the ratio between the loss modulus (G″) and the storage modulus (G′). Strain tests were performed from 0.1 to 100% strain at 1.0 Hz with an equilibration time of 5 min.

### HTST (High Temperature Short Time) Flavored Milk Evaluation

Pasteurized flavored chocolate milk were made using the inventive samples and comparative example 3 described above. The flavored milk formulation consisted of 11.5 wt % full fat milk powder, 7.5 wt % sugar, 0.9 wt % cocoa powder, 0.35 wt % of stabilizer and water to make 100%. The procedure is as follows:
The sugar, cocoa powder and co-processed MCC of the current invention/comparative examples were dry blended.
The pre-blended dry ingredients were added into reconstituted milk (full fat milk powder pre-hydrated for 5 mins) and mixed at medium shear with a propeller mixer for 30 min;
The mixture was pre-heated to 85° C. for 15 sec;
Downstream homogenization was performed at a total pressure of 200 bar (50 bar second stage and 150 bar first stage);
The chocolate milk was then cooled immediately to <20° C. and filled in sterile Nalgene bottles in a clean fill hood.

Samples were evaluated after two weeks of storage at refrigeration temperature (4° C.). Viscosity, pH, phase separation, flow properties, flocculation and dusting level were measured and/or characterized.

### UHT (Ultra High Temperature) Vanilla Flavored Coffee Creamer Evaluation

The tested coffee creamer was formulated as follows: 33% by weight of the creamer of sugar, 10% sunflower oil, 1% sodium caseinate, 0.2% vanilla, 0.23% citrate and phosphate buffer systems, 0.6% mono- and di-glycerides, 0.35% of the stabilizer composition of the invention, and water to make 100%. Comparative tests were made with stabilizer compositions comprising commercial colloidal MCC containing CMC plus carrageenan (0.21%+0.015%), and co-processed colloidal MCC containing CMC plus carrageenan (0.35%+0.015%). The test method is as described below:

The stabilizer sample was dry-blended with sugar and salts.
I. The dry-blend was added to water pre-heated to 80° C.
II. The sunflower oil and emulsifier were added and mixed with a Silverson mixer for 3 min at 4000 rpm. The mixture was cooled to 60° C. and the pH adjusted to 6.7+/−0.05.
III. The mixture was UHT treated at 140° C. for 6 sec.
IV. Downstream homogenization was performed at 180/30 bar at 75° C.
V. Coffee creamers were then cooled immediately to <20° C. and filled in a sterile Nalgene bottle in a clean fill hood.

### Comparative Example 1

MCC/refined Na-iota carrageenan=80/20 was co-processed in a twin-screw extruder. Poor co-attrition was obtained evidenced by slippery texture of the extrudate, low amperage readings (2.8) and low temperatures of extrudate (34.2° C.). No improvement in co-attrition effectiveness was achieved after several passes. This sample was discontinued due to poor quality.

### Comparative Example 2

MCC/E. spinosum flour=80/20 was co-processed in a twin-screw extruder. High amperage readings (5.2), high temperature of extrudate (76.1° C.) and no evidence of slippery conditions were observed. The same extrudate was slurried at 5% in ambient deionized water and then spray dried. The dried powder sample was slurried at 2.6% in deionized water. However, the dispersion was unstable and there was immediate phase separation (water on top and seaweed particles at the bottom). No viscosity measurements were made. This sample was discontinued due to poor functionality.

### Comparative Example 3

MCC/CMC=85/15 was co-processed in a twin-screw extruder. This reference sample recorded an amperage reading of 2.7, and a temperature of 35.9. The same extrudate was slurried at 5% in ambient deionized water and then spray dried. The dried powder sample was slurried at 2.6% in deionized water. The initial viscosity was 430 mPa·s and the 24-hour viscosity was 1690 mPa·s. A colloidal content of 95.32% was obtained. The elastic modulus (G′) was 3.9 Pa.

### Comparative Example 4

A traditional commercial colloidal MCC containing CMC (Avicel® CL611).

### Comparative Example 5

A best-in-class colloidal MCC containing CMC only, under the tradename Avicel®

### Example 1

MCC/E. cottonii flour=85/15 was co-processed in aa twin-screw extruder. The amperage was 4.4 and the temperature of the extrudate was 64.93° C. The same extrudate was slurried at 5% in ambient deionized water and then spray dried. The dried powder sample was slurried at 2.6% in deionized water. The initial viscosity was 250 mPa·s and the 24-hour viscosity was 470 mPa·s. A colloidal content of 13.27% was obtained.

### Example 2

MCC/E. cottonii flour=85/15 was co-processed in a twin-screw extruder. The amperage was 4.4 and the temperature of the extrudate was 64.93° C. The same extrudate was slurried at 5% in ambient deionized water, homogenized and then spray dried. The dried powder sample was slurried at 2.6% in deionized water. The initial viscosity was 200 mPa·s and the 24-hour viscosity was 240 mPa·s. A colloidal content of 12.51% was obtained.

### Example 3

MCC/E. cottonii flour=80/20 was co-processed in a twin-screw extruder. The amperage was 5.2 and the temperature of the extrudate was 63.6° C. The same extrudate was slurried at 5% in ambient deionized water and then spray dried. The dried powder sample was slurried at 2.6% in deionized water. The initial viscosity was 78mPa·s and the 24-hour viscosity was 1700 mPa·s. A colloidal content of 17.63% was obtained. The elastic modulus (G′) was 16.23 Pa.

Table 1 summarizes the HTST chocolate low fat milk evaluation results with comparative example 3, and inventive samples 1-3.

TABLE 1
HTST chocolate milk evaluation results
after 2-weeks storage at 4° C.

| | Inventive | Inventive | Inventive | Comparative |
|---|---|---|---|---|
| | sample 1 | sample 2 | sample 3 | sample 3 |
| Stabilizer | 0.35% | 0.35% | 0.35% | 0.35% |
| Carrageenan | | | | 0.015% |
| Brookfield viscosity, | 51.90 | 46.40 | 78.20 | 654.30 |
| mPa · s | | | | |
| pH | 6.38 | 6.60 | 6.84 | 6.90 |
| Creaming | 0 | 0 | 2 | 0 |
| Serum separation | 0 | 0 | 0 | 0 |
| Marbling | 1 | 2 | 1 | 0 |
| Top Phase separation | 11 mm | 17 mm | 0 | 0 |
| Flow property | 0 | 0 | 0 | 3 |
| (gelation) | | | | |
| Sedimentation layer | 0 | 0 | 0 | 0 |
| Re-dispersibility | 0 | 0 | 0 | 0 |
| Flocculation | 0 | 0 | 0 | 0 |

The results of table 1 shows acceptable functionality of inventive sample 1 compared to comparative example 3. Inventive sample 3 had acceptable viscosity of beverage and no gelation. Comparative sample 3, however, showed a high viscosity and unacceptable gelation, conditions which may help describe the beverage as heavy. Thus, inventive sample 3 shows superior performance.

Tables 2 summarizes the UHT coffee creamer evaluation results with comparative examples 3-5, and inventive samples 2 and 3.

TABLE 2
1-month evaluation results of vanilla flavored coffee creamers stabilized
by inventive samples and comparative samples. Samples were stored at 4° C.

| | Inventive | Inventive | Comparative | Comparative | Comparative |
|---|---|---|---|---|---|
| Sample No. | sample 3 | sample 2 | sample 3 | sample 4 | sample 5 |
| Dose Level, % | 0.35% | 0.35% | 0.35% + 0.015% | 0.35% + 0.015% | 0.21% + 0.015% |
| | | | CGN | CGN | CGN |

Before any manipulation

| | | | | | | |
|---|---|---|---|---|---|---|
| Top phase separation | UHT, | 4-5 mm | 3 mm | 9 mm | 5 mm | 2 mm |
| (Creaming) | Chilled | | | | | |
| Serum separation or- Clear | | 0 | 0 | 0 | 0 | 0 |
| water phase | | | | | | |
| Marbling (Clear layers of whey | | 0 | 0 | 2 | 1-2 | 0 |
| inside product) | | | | | | |
| Syneresis (Water separation | | 0 | 0 | 0 | 0 | 0 |
| from gel network) | | | | | | |
| Sedimentation | | 0 | 0 | 0 | 0 | 0 |
| During pouring | | | | | | |
| Gelation (liquid/gel) | | 0-1 | 0 | 0-1 | 0-1 | 0 |
| Viscosity - 30 RPM | | 164.40 | 82.80 @ 30 rpm | 141 | 41 | 50 |
| Pours | | 0 | 0 | 0 | 0 | 0 |
| Sedimentation at bottom | | 0 | 0 | 0 | 0 | 0 |
| Flocculation of proteins | | 0 | 0 | 0 | 0 | 0 |
| PH | | 6.86 | 6.89 | 6.87 | 6.81 | 6.81 |

CGN: carrageenan

Inventive sample 2 was most similar in physical stability to comparative example 5 (a product described as a′best-in-class' colloidal MCC for this type of application), but with a higher viscosity as expected. At the sample scale of development, comparative example 3 experienced pronounced phase separation compared to the inventive samples. Viscosities of Inventive sample 3 matched comparative example 3 as expected. Thus, table 2 shows comparable performance of the inventive samples compared to the ‘best-in-class’ stabilizer, and a superior performance to a traditional colloidal MCC stabilizer.

We have developed colloidal MCC products without CMC and attrition aids in this invention. The disclosed stabilizers are capable of superior stabilization of dairy beverages compared to stabilizers containing MCC and CMC. These unexpected findings are observed despite the use of unrefined red seaweed flour compared to CMC or refined carrageenan.

1. A stabilizer composition comprising colloidal microcrystalline cellulose coprocessed with unrefined and unmodified red seaweed flour derived from a red seaweed of the class Rhodophyta.

2. The stabilizer composition of claim 1, wherein the red seaweed flour is derived from red seaweed of the class Rhodophyta that is rich in kappa-carrageenan and low in iota-carrageenan.

3. The stabilizer composition of claim 2, wherein the red seaweed flour comprises kappa-carrageenan in an amount of 25-70 wt % by dry weight of the red seaweed flour.

4. The stabilizer composition of claim 3, wherein the red seaweed flour comprises kappa-carrageenan in an amount of 40-70% by dry weight of the red seaweed flour.

5. The stabilizer composition of claim 2, wherein the red seaweed flour comprises less than 10% iota-carrageenan by dry weight of the seaweed flour.

6. The stabilizer composition of claim 2, wherein the red seaweed flour is derived from the Rhodophyta genera Kappaphycus, Eucheuma, Gigartina, Chondrus, Iriadae, Mazzaella, Mastocarpus, Sarcothalia, Hypnea, Furcellaria, Gracilaria, Gelidium, Gelidiella. Pterocladia, Halymenia or Chondracanthus, preferably the species Euchema striatum/Kappaphycus striatus, Euchema alvarezii/Kappaphycus alvarezii, or a combination thereof such as Eucheuma/Kappaphycus cottonii.

7. The stabilizer composition of claim 1, wherein the weight ratio of colloidal microcrystalline cellulose to red seaweed flour is between 70:30 and 90:10, preferably between 80:20 and 85:15.

8. The stabilizer composition according to claim 7, wherein the kappa-carrageenan in the non-coprocessed red seaweed flour has a weight average molecular weight of from 900 to 2000 kDa, preferably from 1000 to 1500 kDa.

9. The stabilizer composition of claim 1 which has an initial viscosity of 50-1000 mPa·s when measured as a 2.6% by weight dispersion in deionized water on a Brookfield RV viscometer, spindle #3, at 20 rpm and 20° C.

10. The stabilizer composition of claim 1 which has a 24-hour viscosity of 150-5000 mPa·s when measured as a 2.6% by weight dispersion in deionized water on a Brookfield RV viscometer, spindle #3, at 20 rpm and 20° C.

11. A process for preparing the stabilizer composition of claim 1, comprising the steps of
(a) blending a microcrystalline cellulose wetcake with red seaweed flour derived from a red seaweed of the class Rhodophyta,
(b) co-processing the blend of step (a) in the absence of an attrition aid to form an extrudate, and
(c) drying and optionally milling the extrudate to a powder.

12. The process of claim 11, wherein the extrudate of step (b) is homogenized before drying in step (c).

13. The process of claim 11, wherein an additional amount of red seaweed flour is blended with the extrudate of step (b) before drying in step (c) or with the dried and milled extrudate of step (c).

14. An edible product comprising the stabilizer composition of claim 1.

15. The edible product of claim 13, which is a beverage product.

16. The edible product of claim 15 which is a dairy milk beverage or a plant protein beverage.

17. The edible product of claim 14 which is a dairy or non-dairy creamer.

18. The edible product of claim 14 comprising 0.1-0.50% by weight of the stabilizer composition.

19. The edible product of claim 18 comprising 0.20-0.35% by weight of the stabilizer composition.
